(19) United States
(12) Patent Application Publication
(10) Pub. No.: US 2010/0273157 A1
(43) Pub. Date: Oct. 28, 2010

(54) System and Method for Authentication or Identification of Bio-Artifacts Related to President Abraham Lincoln

(76) Inventor: John George Sotos, Palo Alto, CA (US)

Correspondence Address: John G. Sotos, 1788 Oak Creek Dr., #415, Palo Alto, CA, 94304, US

(21) Appl. No.: 12/619,699

(22) Filed: Nov. 17, 2009

Related U.S. Application Data
(60) Provisional application No. 61/199,426, filed on Nov. 18, 2008.

(51) Int. Cl. C12Q 1/68

(52) U.S. Cl. 435/6

(57) ABSTRACT

An invention for authenticating artifacts related to President Abraham Lincoln is presented. In an embodiment of the invention, an artifact is identified. The artifact has a biologically-derived component (“bio-component”). At least a portion of the bio-component is processed to yield matter that may be analyzed. This matter is analyzed and the analysis results interpreted. The interpretation is based on information related to a syndrome of marfanoid habitus and stable multiple lip dysmorphisms (an “MH/LD syndrome”). Two such syndromes are multiple endocrine neoplasia type 2B and pure mucosal neuroma syndrome. Other such syndromes may exist. Evidence of an MH/LD syndrome in the analysis results is evidence that the artifact is authentically associated with President Lincoln. A report is optionally produced.

### CROSS-REFERENCES TO RELATED APPLICATIONS

This application claims priority to U.S. Provisional Patent No. 61/199,426, titled “System and Method for Authentication or Identification of Bio-Artifacts Related to Abraham Lincoln,” filed on 18 Nov. 2008, hereby incorporated by reference for all purposes.

### BACKGROUND OF THE INVENTION

It is well known that some artifacts associated with a particular person (herein generically called a “celebrity,” with no defined minimum level of fame) may carry great monetary or other value. For example, in 2008, a portion of a shirt collar, stained with blood said to be that of President Abraham Lincoln (Heritage Auctions Inc.; M. Dixey (ed.). Heritage Historical Americana Auction #6014 the Dr. John K. Lattimer Collection of Lincolniana. Dallas, Tex.: Heritage Capital Corporation, 2008), fetched a bid over $65,000 at auction (Heritage Auctions Inc. “Auction Archives.” Web page downloaded from http://historical.ha.com on 19 Apr. 2009).

Because some artifacts may be counterfeit, or may be incorrectly associated with a celebrity, collectors of artifacts (to name just one group of potentially interested parties) often desire evidence illuminating the truth or falsity of claims about the artifact. In particular, they may desire evidence that the artifact is genuine, i.e. is truly associated with the claimed celebrity. In the case of the shirt collar, above, for example, it is reasonable to believe it would have attracted a lower auction bid if strong evidence emerged that the blood on the collar was not Abraham Lincoln's.

Authenticating the historical background of an artifact may sometimes be problematic. For example, statements from the artifact owner, or from previous owner(s), may be offered as evidence supporting claims about the artifact's historical background, but, in many cases, such statements fail to authenticate the historical background of the artifact with a desired level of confidence. Merely by way of example, the reasons for this failure may include little or no independent or external support for the statements, gaps in the artifact's history, doubts about the trustworthiness or accuracy of a statement's sources, or other factors.

Some artifacts are amenable to other methods of authentication. For example, some artifacts—herein called “bioartifacts”—contain a biologically-derived component. In some cases, analysis of the biologically-derived component of a bioartifact can contribute to authentication of the bioartifact. Biologically-derived components include, but are not restricted to DNA, RNA, protein, and various other components of bodily fluids, tissues, secretions, excretions, and so on.

It is often possible to make a claim relating a bioartifact and a celebrity. Some such claims relate to the bioartifact's genuineness, i.e. the probability that at least a portion of the bioartifact derives from biochemical elements of the celebrity. Herein, “authentication” will refer to establishing or revising a probability that a positive claim about a bioartifact and a celebrity is true. Thus, authentication need not be definitive (it may result in probability values other than 0% and 100%), and depending on its outcome it may increase or decrease or leave unchanged the genuineness probability. A concept of “positive authentication” exists (increased probability that the artifact and the celebrity are associated), as does a concept of “negative authentication” (decreased probability that the artifact and the celebrity are associated).

In some cases, evidence pertaining to the genuineness of a bioartifact may derive from analysis of its biochemical component(s). For example, a bioartifact may include a bloodstain as a biochemical component. In principle, DNA in the bloodstain could be used to confirm or disconfirm a particular person or group of people as the source of the DNA and, hence, as the source of the biochemical component of the bioartifact.

A plurality of factors may complicate the authentication analysis of some bioartifacts, however.

(1) The biochemical material may have degraded (especially if it is old).
(2) The biochemical material may be in short supply.
(3) Access to biochemical material may be restricted.
(4) There may be no standards by which the biochemical material in the bioartifact can be associated with a particular individual.
(5) The biochemical material may be contaminated with biochemical material from other organisms.

Consider, for example, the case of President Abraham Lincoln. (Several bioartifacts related to President Lincoln are listed in a book: Sotos J G. The Physical Lincoln Sourcebook. Version 1.1a. Mt. Vernon, Va.: Mt. Vernon Book Systems, 2008, especially paragraphs 3246-3264. This book is incorporated by reference for all purposes in its entirety, and is herein referred to as “PLS” or “The Physical Lincoln Sourcebook.”) The following considerations, among other possible ones, appear to apply to some bioartifacts related to President Lincoln:

Consideration #1: The biochemical material is old and may be degraded.—President Lincoln died in 1865, meaning no new biochemical components of his have been made by his living processes since then. Thus, whatever such biochemical components were extant at his death have had more than 144 years to degrade. Degraded biochemical components are often more difficult to analyze accurately. For example, in the case of DNA, strand breakage, de-purination, and destruction of the ribose ring may occur over time (F A Kaestle and K A Horsburgh. “Ancient DNA in anthropology: methods, applications, and ethics.” Yearbook of Physical Anthropology. 2002; 45: 92-130.)
Consideration #2: The biochemical material may be in short supply.—For example, a “small strip of linen” is reputed to be stained with President Lincoln's blood (H. Levins. “Keeping Abraham Lincoln's blood on file in Camden: curious relics in the County Historical Society's Civil War Collection.” Dec. 20, 2004; web page downloaded from http://historiccamdencounty.com/ccnews92.shtml on Apr. 16, 2009). Compared to say, a 7 ml vial of blood, the amount of biochemical material (e.g. DNA) on such a linen strip would be far less.
Consideration #3: Access to biochemical material may be restricted.—Typically, some biochemical components cannot be conveniently or reliably analyzed unless they are separated from the artifact—a process that may result in defacement or otherwise disturb the artifact. Many artifacts connected to President Lincoln are classified as “historic” and some owners of these artifacts might not wish to see the artifact defaced or otherwise disturbed. Even when an owner allows some disturbance of an artifact, it might be that the owner would wish the disturbance limited, which, in turn, could limit the amount of biochemical material for analysis. For example, Reilly teaches that access to fragments of President Lincoln's skull for the purpose of biochemical analysis became the concern of a government committee in the 1990s (P. R. Reilly. Abraham Lincoln's DNA and Other Adventures in Genetics. Cold Spring Harbor, N.Y.: Cold Spring Harbor University Press, 2000. Pages 2-13).

Given the above, it is reasonable to conclude that even biochemical analyses that often require small amounts of material (e.g. DNA analysis) may still demand more material than is available once degradation, quantity, access, and/or potentially other factors are considered.

For example, Krawczak and Schmidtke teach: “Using the widely accepted estimate that two homologous chromosomes randomly drawn from the human genome differ at a frequency of 1 in 300 by [base pairs], sequencing a 15 000 by segment would guarantee that, with 99.9% probability, no pair of unrelated humans living on earth would be found to be identical” (M. Krawczak, J. Schmidtke. DNA Fingerprinting. 2nd edition. New York: Springer-Verlag (in cooperation with BIOS Scientific Publishers, Oxford, UK), 1998, page 2). In some cases it may be difficult to gain access to 15,000 base pairs of analyzable DNA in a bioartifact. It is reasonable to expect that, in general, the more base pairs of analyzable DNA required by an authentication technique, the more likely it is that factors such as degradation, quantity, and access could up to a point interfere with successful application of a technique. Authentication techniques based on smaller lengths of DNA would, therefore, seem to have advantages in many cases.

Additional considerations for authentication analyses of bioartifacts include, but are not restricted to:

Consideration #4: There may be no standards by which the biochemical material in the bioartifact can be associated with a particular individual.—If, for example, analysis of an alleged Lincoln bioartifact yields a DNA sequence, how can one know if this DNA sequence is President Lincoln's, as opposed to someone else's? Although DNA and RNA may be potentially attractive biochemical materials for authenticating bioartifacts (in part because very few people are genetically identical), using them to authenticate bioartifacts related to President Lincoln is challenging today because no nucleic acid sequence(s) having low prevalence in the general population have yet been publicly associated with President Lincoln with a high degree of certainty.

One potential way to overcome this “standards” issue would be to adopt as a standard the biochemical signature from a bioartifact unequivocally known to contain biochemical material from President Lincoln. Finding such unequivocal artifacts may, however, prove difficult. For example, Reilly (supra.) teaches “tissue known to be derived from the President [Lincoln] (because the samples have been in the museum [National Museum of Health and Medicine] since the assassination),” but Davidson teaches that “the bone, hair, and blood-stained cuffs from Edward Curtis, the doctor performing the autopsy, did not come into the museum's collections until Sep. 12, 1947” (G. W. Davidson. “Abraham Lincoln and the DNA controversy.” Journal of the Abraham Lincoln Association. 1996; 17(1): 1-26, as web page downloaded on Oct. 31, 2009 from: http://www.historycooperative.org/journals/jala/17.1/davidson.html)—thus raising questions about the provenance and genuineness of these supposed “unequivocal” artifacts. Similar questions would likely attend other proposed “standards” sources.

In the case of authenticating a putative Lincoln-related bioartifact, certain characteristics of Lincoln could be sought in the biological component of the artifact. For example, Lincoln was male, so evidence of male-ness (e.g. a DNA sequence associated with a Y-chormosome, or gene expression profiles compatible with a male hormone pattern) could be assessed in the bioartifact. However, because male-ness is common in the world, finding evidence of male-ness in a bioartifact would not ordinarily give great confidence that a bioartifact was associated with Lincoln.

Similarly, it has been reported that Lincoln had blood type A (M. S. Micozzi. “When the patient is Abraham Lincoln.” Caduceus. Spring 1991; 7: 34-43). A putative Lincoln-related bioartifact could, therefore, be tested for evidence of blood type A (e.g. the corresponding protein or DNA sequence(s) associated with blood type A). However, blood type A is also common in the world, so, as with maleness, a positive result would not give great confidence that the bioartifact was associated with Lincoln. Moreover, the certainty of the claim that Lincoln had blood type A is not clear from Micozzi's article.

Analyzing a bioartifact for both male-ness and blood type A could be done, in principle, but, again, many men have blood type A.

Consideration #5: The biochemical material may be contaminated with biochemical material from other organisms.—For example, if a person (other than Lincoln) were to cough on a bioartifact reputed to be associated with Lincoln, it is possible that some DNA in the cougher's cough droplets would become deposited on the artifact. Biochemical analysis of the artifact afterwards could, therefore, yield results not from Lincoln, but from the cougher.

Contamination can sometimes lower the signal-to-noise ratio of an analysis. More subtle effects may also occur, however. For example, suppose an authentication technique depends on two findings, labeled “A” and “B” in this example, that both occur in President Lincoln's DNA, but rarely occur together in the DNA of other humans. Suppose, too, it is not unusual for A to occur singly in human DNA, and for B to occur singly in human DNA. In this situation, an artifact that was contaminated by DNA from one person having the “A” finding and also contaminated by DNA from a second person having the “B” finding, might (incorrectly) appear to contain both A and B when analyzed. This would increase the unreliability of the “A and B” authentication technique.

Many of the same or similar factors noted above also apply to bioartifacts related to President Lincoln's family members.

From that above, it is desirable to have improved techniques of authenticating bioartifacts related to Abraham Lincoln and his family.

### BRIEF SUMMARY OF THE INVENTION

An invention for authenticating bioartifacts related to President Abraham Lincoln and possibly some of his family members is presented.

In an embodiment of the invention, an artifact is identified. The artifact has a biologically-derived component (“bio-component”). At least a portion of the bio-component is processed to yield matter that may be analyzed. This matter is analyzed. Results of the analysis are interpreted. The artifact or bio-component has an associated claim about an association between the artifact (or bio-component) and a person of interest. The claim is based on evidence derived from source(s) other than the analysis and interpretation. The claim is associated with a first probability of being true. The interpretation leads to revision of the first probability to a second probability that the claim is true. A report is optionally produced.

The analysis and/or interpretation is based on information related to a syndrome of marfanoid habitus and stable multiple lip dysmorphisms (an “MH/LD syndrome”). Two such syndromes are multiple endocrine neoplasia type 2B and pure mucosal neuroma syndrome. Other such syndromes may exist.

In some cases the person of interest is President Abraham Lincoln or a blood relative of his. If the claim associates the bioartifact with President Lincoln, and if the analysis yields biochemical evidence of an MH/LD syndrome in the bioartifact, then the second probability is generally higher than the first probability.

Other embodiments of the invention are possible.

It should be noted that the above sequence of steps is merely illustrative. Any of the above steps can also be separated or combined, depending upon the embodiment. In some cases, the steps can also be changed in order without limiting the scope of the invention claimed herein. One of ordinary skill in the art would recognize many other variations, modifications, and alternatives. It is also understood that the examples and embodiments described herein are for illustrative purposes only and that various modifications or changes in the light thereof will be suggested to persons skilled in the art and are to be included within the spirit and purview of this document.

### BRIEF DESCRIPTION OF THE DRAWINGS

FIG. 1 shows a flowchart of an embodiment of the invention.

FIG. 2 shows a flowchart of an embodiment of the invention.

FIG. 3 shows a flowchart of an embodiment of the invention.

### DETAILED DESCRIPTION OF THE INVENTION

The genetic health of the late 16th President of the United States, Abraham Lincoln (sometimes called just “Lincoln” herein), has been discussed by physicians since at least 1962, when Dr. Abraham Gordon published an article considering the possibility that Lincoln had the genetic condition called Marfan syndrome (A. M. Gordon. “Abraham Lincoln—a medical appraisal.” J Ky Med Assoc. 1962; 60: 249-253). Other physicians disagree with this diagnosis (e.g. J. K. Lattimer. “Lincoln did not have the Marfan syndrome.” NY State J Med. 1981; 81: 1805-1813).

As is known to persons skilled in the art, a common feature of Marfan syndrome is “marfanoid habitus.” Marfanoid habitus may be conceptualized as referring to a particular body shape that is commonly seen in persons with Marfan syndrome, the shape including but not restricted to tallness, long limbs, and slender build. Gordon (supra.) teaches that Lincoln had many elements of a marfanoid habitus; Lattimer (supra.) teaches that Lincoln did not. Applicant believes that Lincoln did have a marfanoid habitus.

Marfanoid habitus is associated with other medical conditions besides Marfan syndrome. At least two, Stickler syndrome and the MASS phenotype, have been proposed as diagnoses for Lincoln.

In attempting to reach a diagnosis for Lincoln, applicant has given substantial weight to Lincoln's body shape and to his lips (J. G. Sotos, The Physical Lincoln. Version 1.1a. Mt. Vernon, Va.: Mt. Vernon Book Systems, 2008, pages 128-135; henceforth abbreviated TPL or The Physical Lincoln; incorporated by reference for all purposes in its entirety).

Hamilton and Ostendorf teach that Lincoln had an “odd lump” on the right lateral aspect of his lower lip (C. Hamilton, L. Ostendorf. Lincoln in Photographs: An Album of Every Known Pose. Norman, Okla.: University of Oklahoma Press, 1963, page 163). Applicant has discovered that photographs of Lincoln show what appear to be more than one one bump on (or in) his lips (TPL, page 128). Applicant has also discovered that the bumps on or in Lincoln's lips do not substantially change in photographic appearance over a period of years (TPL, page 128), subject to limitations of the photographs available to applicant.

Acquaintances of Lincoln have described his lower lip as “thick” or “somewhat thick” (TPL, p 132). Photographs available to applicant appear to show that Lincoln's lower lip was thick from the time of his earliest known photograph (taken about 1846) to the year of his death, 1865 (TPL, page 133). A textual account suggests that Lincoln's lower lip was prominent even in his youth (TPL, pages 131-132).

A variant in the size or shape of a lip that does not substantially change over a sustained period of time may be termed a “stable lip dysmorphism.” By this definition, Lincoln's lips had multiple stable lip dysmorphisms. The plurality of stable lip bumps, noted above, alone satisfies the definition. In some cases, stability of lip dysmorphisms can aid diagnosis by eliminating disorders, such as mild infections or mild trauma, that generally cause transient changes in lip size or shape.

A syndrome that is associated with the co-existence of marfanoid habitus and multiple stable lip dysmorphisms may be termed “an MH/LD syndrome.” MH/LD syndromes include multiple endocrine neoplasia type 2B (MEN2B) and the pure mucosal neuroma syndrome (PMNS) (G. Spyer et al. “Phenotypic multiple endocrine neoplasia type 2B, without endocrinopathy or RET gene mutation: implications for management.” Thyroid. 2006; 16: 605-608). Other MH/LD syndromes may exist. MEN2B has also been called multiple endocrine neoplasia type 3, Wagenmann-Froboese syndrome, and other names.

Applicant has discovered that Lincoln's phenotype is compatible with an MH/LD syndrome, and that it is most compatible with MEN2B (Sotos, supra., The Physical Lincoln).

Hoff and Gagel teach that “MEN2B is an autosomal-dominant genetic syndrome that includes MTC [medullary carcinoma of the thyroid], pheochromocytoma, multiple mucosal neuromas, and a marfanoid habitus” (A. O. Hoff, R. F. Gagel. “Multiple endocrine neoplasia type 2.” Pages 3533-3550 (Chapter 192) in: L. J. DeGroot, J. L. Jameson (eds). Endocrinology. 5th ed. Philadelphia: Elsevier-Saunders, 2006) Hoff & Gagel (supra.) teach that MEN2B is but one of the MEN2 syndromes (MEN2A being another) and add “MEN2-related syndromes are uncommon, with probably fewer than 1500 kindreds worldwide” (Hoff & Gagel, supra.). Given a world population on the order of 6.8 billion, and given that MEN2B is said to constitute a minority of MEN2 cases (B. A. J. Ponder. “Multiple endocrine neoplasia type 2.” Pages 931-942 (Chapter 42) in: Scriver C R; Beaudet A L; Sly W S; et al. The Metabolic and Molecular Basis of Inherited Disease. 8th ed. New York: McGraw-Hill, 2001), it is reasonable to use a prevalence figure of 1 in a million for MEN2B, recognizing that it is a crude approximation.

Table 1 shows correspondences between some features of Lincoln's medical history and some features of MEN2B. The Physical Lincoln (supra.) contains further information. Table 1 notes that a marfanoid habitus is a (common) feature of MEN2B. It has been reported that in many cases of MEN2B, the lips appear large (the word “blubbery” has been used to describe them) and bumpy.

Although the correspondences in Table 1 may appear robust, the diagnosis of MEN2B for Lincoln was not obvious, even to those skilled in the art. Indeed, some of Lincoln's features may be considered atypical for the disease, as it is currently understood (TPL, pages 135, 176-179). For example, photographs show that Lincoln's upper lip was not greatly enlarged. As a second example, Lincoln's survival to age 56 without treatment is unusual in MEN2B.

It appears that MEN2B (or any MH/LD syndrome) was not suggested as a diagnosis for Lincoln before the applicant did so, despite the thousands and thousands of books and articles written about him, and despite medical analyses by geneticists (e.g., R. Marion. “Was George Washington Really the Father of Our Country?” Reading, Mass.: Addison-Wesley, 1994. Pages 88-124) (e.g., V. McKusick. “Abraham Lincoln and Marfan Syndrome.” Nature. 1991; 352: 280), other physicians (e.g., Gordon. supra.) (e.g., H. Schwartz. “Abraham Lincoln and the Marfan syndrome.” JAMA. 1964; 187: 473-479), and historians (e.g., G. S. Boritt, A. Borit, “Lincoln and the Marfan syndrome: the medical diagnosis of a historical figure.” Civil War History. 1983; 29: 212-229). Dr. V. McKusick was late chairman of the Department of Medicine at Johns Hopkins, and was recognized by many as a leading authority on Marfan syndrome and other heritable disorders of connective tissue.

Applicant has also discovered that President Lincoln was not the only person in his family who likely had MEN2B (TPL, page 179). Table 2 shows other blood-relatives of his who have a high probability of having had MEN2B according to the material presented in The Physical Lincoln. The earliest-born identifiable person listed in Table 2 is President Lincoln's biological mother, Nancy Hanks Lincoln. If she had MEN2B, it is possible that it arose in her as a spontaneous genetic mutation, or that she inherited it from her father. (It is unlikely that her mother, Lucey Hanks, had MEN2B, given that Lucey Hanks is said to have survived to age 66 (PLS, page 380).) Herein, a person with a “high probability” of having MEN2B is defined to mean that (a) the person had, or could have had, one or more specific features of MEN2B, without feature(s) strongly contravening MEN2B, and (b) the person likely had a blood relationship to Nancy Hanks Lincoln, with no one other than high-probability-MEN2B-people interposed.

A parsimonious hypothesis for the pattern of disease reflected in Table 2 is that Nancy Hanks Lincoln passed an MEN2B-conferring gene from herself to her son Abraham, and he in turn passed it to three of his sons. Any role of the father of Nancy Hanks Lincoln in passing an MEN2B-conferring gene is currently unclear.

The list of President Lincoln's blood relatives who are defined as having a high probability of MEN2B may expand and contract according to later discoveries. For example, it is generally acknowledged that the identity of the father of Nancy Hanks Lincoln is today unknown (M. Burlingame, Abraham Lincoln: A Life. Baltimore: Johns Hopkins University Press, 2009). Should this man's identity some day become known, facts about his life may enable a conclusion that he was not afflicted with MEN2B, and he would drop off the list. Alternatively, facts about this man's life could establish that he was likely afflicted with MEN2B and, further, that one of his parents was, too, so that this afflicted parent (i.e. a paternal grandparent of Nancy Hanks Lincoln) would then be added to the list.

Hoff and Gagel (supra.) teach that MEN2B occurs “as a result of mutations of the RET proto-oncogene.” Table 3 lists some germ line variations in the RET gene that have been associated with MEN2B. Ponder (supra., page 936) teaches that “some 95 percent of MEN 2B families reported to date” have the M918T variant in the RET protein. Table 3 may change over time. Hoff & Gagel (supra.) teach that approximately one new MEN2 DNA variant per year is discovered.

Given a diagnosis of MEN2B for President Lincoln, and given the relationship between MEN2B and RET, it is reasonably concluded that Lincoln likely had a germ line variant in his germ line RET gene. (The same reasoning would likely apply to other persons listed in Table 2.) The variant may be from Table 3, or it may be a gene variant (in RET or another gene(s)) that has heretofore not yet been related to the clinical syndrome of MEN2B but is later shown to be related.

The conclusion that RET variations are present with high probability in at least one person in Table 2 is incorporated into an invention for authenticating bioartifacts claimed to derive from a Table 2 person (“Table 2 person” meaning a person meeting the criteria for inclusion in Table 2).

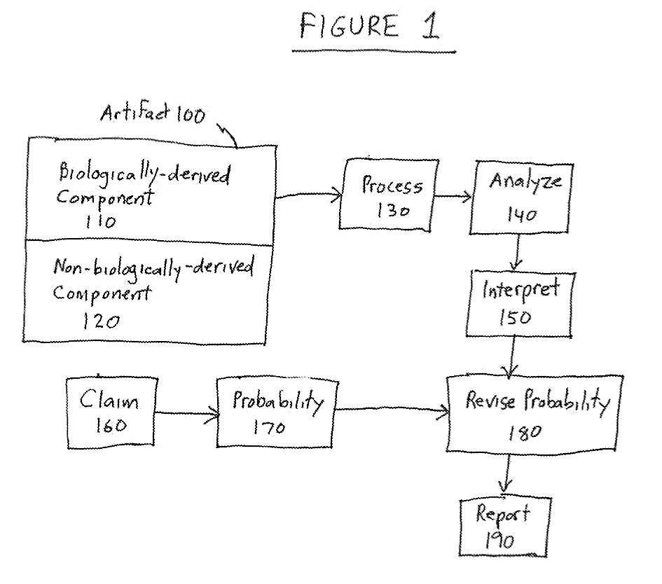

FIG. 1 shows an embodiment of the invention. Artifact 100 has a biologically-derived component 110 (also called “bio-component”). Artifact 100 may or may not have a component 120 that is not biologically derived. Bio-component 110 is processed 130 to yield matter that may be analyzed 140. Results of analysis 140 are interpreted 150. Artifact 100 or bio-component 110 has an associated claim 160. Claim 160 is a claim for an association between artifact 100 (or bio-component 110) and a person of interest (not shown in FIG. 1). Claim 160 is based on evidence derived from source(s) other than analysis 140 and interpretation 150. Claim 160 is associated with a probability 170 of being true. Interpretation 150 leads to revision 180 of probability 170 that claim 160 is true. Report 190 is optionally produced. Contents of report 190 may include, but are not restricted to: results of analysis 140, results of interpretation 150, revised probability 180.

Merely by way of example, blood, hair, bone, brain matter, saliva, sweat, among other substances, are biologically derived. Presence or absence of bio-component 110 associated with artifact 100 may not be apparent until after subsequent steps in the invention. For example, if a gun was possibly handled by Lincoln, it may be uncertain whether any biological material derived from Lincoln (or anyone else) was left on the gun after the handling. In this example, processing 130 and/or analysis 140 might demonstrate the presence of biologically-derived material on the gun that was not apparent before those step(s).

Processing 130 may include, but is not restricted to, obtaining a sample of bio-component 110, e.g. pulling a few blood-stained threads from a blood-stained piece of fabric, cutting away a blood-stained piece of fabric from a larger piece, selecting a subset of hairs from a group of hairs, chipping away a piece of bone from a larger bone, rubbing a bit of dried blood off of a blood-stained piece of paper, etc. Processing 130 may include, but is not restricted to, extraction of DNA, amplification of DNA, other chemical or physical procedures, etc. (Kaestle & Horsburgh, supra.).

In an additional embodiment of the invention, processing 130 of bio-component 110 is not performed. Merely by way of example, this may occur if bio-component 110 is amenable to analysis 140 in its unprocessed state.

Merely by way of example, information provided by analysis 140 could be derived from analysis of DNA, RNA, protein, other matter, or combinations thereof. Analysis 140 may include, but is not restricted to, application of chemicals, application of electromagnetic radiation, or application of a force to the matter yielded by processing 130 (or to bio-component 110 if processing 130 was not performed). Results of application 140 may be stored, e.g. in computer memory, in a database, on paper, etc.

Analysis 140 may be directed to or more target molecule fragments, the target molecule fragments being indicative of MEN2B or another MH/LD syndrome. For example, analysis 140 may be directed to a specific region in DNA, or to a specific region in RNA, etc.

Interpretation 150 relates the information from analysis 140 to an MH/LD syndrome. Merely by way of example, analysis 140 may provide information that at least some RET proteins in bio-component 110 are the M918T variant. Continuing the example, interpretation 150 would relate this information to a list of protein variants associated with MEN2B (MEN2B being an MH/LD syndrome). Merely by way of example, interpretation 150 may be performed by a human or by a computing device. Merely by way of example, interpretation 150 may be algorithmic or heuristic. Results of analysis 150 may be stored, e.g. in computer memory, in a database, on paper, etc.

Examples of claim 160 include, but are not restricted to, (a) “This piece of fabric is stained with Lincoln's blood,” (b) “This piece of fabric is not stained with Lincoln's blood,” (c) “This piece of fabric is stained with the blood of Mao Zedong,” (d) “This piece of fabric is stained with the blood of someone other than Abraham Lincoln,” (e) “This piece of fabric once was in contact with fluid from Lincoln's body.” (f) “Lincoln touched this piece of paper,” (g) “These hairs are from Nancy Hanks Lincoln,” (h) “This is the corpse of Nancy Hanks Lincoln,” (i) “This bone fragment is from Lincoln's skull,” (j) “This coprolite is from the Lincoln home privy,” (k) “This piece of fabric is stained with the blood of Mao Zedong.” The previous examples demonstrate that claim 160 may in some cases be a claim of positive association (e.g. example (a)) or a claim of negative association (e.g. example (b)). The previous examples also demonstrate that the person of interest referred to by claim 160 may be a Table 2 person (e.g. example (a)) or a person not in Table 2 (e.g. example (c) assuming that Mao Zedong is not eligible for listing in Table 2). The previous examples also demonstrate that claim 160 may refer to a person of interest indirectly (example (j)).

In some cases, a claim about artifact 100 (or bio-component 110) may refer to a plurality of persons, e.g. “This fabric is stained with the blood of one of Lincoln's short-lived children” or “This fabric is stained with the blood of people unrelated to Nancy Hanks Lincoln.” Such claims are understood to contain a plurality of claims 160 about individuals. For example, “This fabric is stained with the blood of one of Lincoln's short-lived children” may be understood to be equivalent to the logical disjunction of three claims 160: “This fabric is stained with the blood of Eddie Lincoln,” “This fabric is stained with the blood of Willie Lincoln,” “This fabric is stained with the blood of Tad Lincoln.” It would of course be daunting to enumerate all the people unrelated to Nancy Hanks Lincoln, but in principle it is possible. A “person group” consists of one or more persons.

Merely by way of example, probability 170 may be quantitative or qualitative (e.g. “high”, “low”, etc.), absolute or relative, expressed or non-expressed, implicit or explicit, objective or subjective, zero or unity (or in-between), be a combination of a plurality of these characteristics, have another characteristic, etc. A person who thinks “The blood-stained piece of pillow-case that I once saw in a museum may have the blood of Abraham Lincoln on it” illustrates an example of probability 170. A computer that has stored in memory a numerical probability for a claim 160 illustrates an example of probability 170. Many other examples of probability 170 are possible.

Probability 170 may be based on the historical background of artifact 100, or on other source(s). Merely by way of example, probability 170 may in some cases depend on whether claim 160 is based on second-hand testimony from a certain person, or is based on an affidavit filed by a surgeon's widow, or is based on family lore, or is based on a location of artifact 100 (e.g. spectacles found in Lincoln's pockets after he was shot), or is based on a note written on cardboard inside a picture frame where artifact 100 is mounted. Other examples are possible.

In some cases, claim 160 and probability 170 may be combined in a single statement, e.g. “It is unlikely that this is a hair from Lincoln's head,” or “It was rumored that Lincoln kissed this Bible,” or “It is possible that this tooth was Willie Lincoln's.”

Revised probability 180 may have characteristics similar to probability 170. Revising probability 170 may be viewed as an authentication function as applied to artifact 100 (or bio-component 110).

Merely by way of example, report 190 may be oral, written, or stored in a computer memory or on a computer-readable medium, possibly connected to a network. Report may contain information related to any of the steps or objects or concepts in FIG. 1. A plurality of reports 190, or portions of reports 190, may be stored, e.g. in a database.

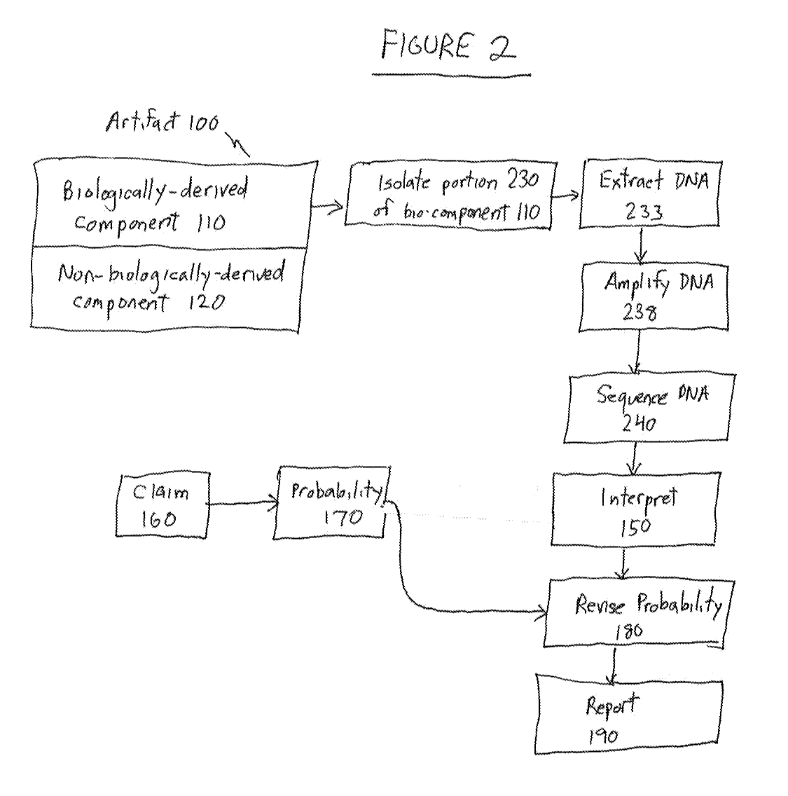

FIG. 2 shows an embodiment of the invention. Artifact 100, bio-component 110, and non-biologically-derived component 120 are as in FIG. 1. A portion 230 of bio-component 110 is isolated. DNA in portion 230 (or in bio-component 110) is extracted 233. Extracted DNA is amplified 238. Amplified DNA is sequenced 240. Interpretation 150, claim 160, probability 170, revised probability 180, and report 190 are as in FIG. 1.

As known to persons skilled in the art, DNA extraction 233 may be performed using a technique based on silica or phenol or using another technique or a combination of techniques (Kaestle & Horsburgh, supra.). In principle, if a technique were available to analyze DNA in situ on the bioartifact, then extraction 233 would not be needed.

As known to persons skilled in the art, DNA amplification 238 may be performed using the polymerase chain reaction (PCR) or using another technique or a combination of techniques (Kaestle & Horsburgh, supra.). Amplification using clonal techniques may, in some cases where bio-component is contaminated with DNA from person(s) other than the person of interest, be desirable. Examples of clonal amplification techniques include, but are not restricted to TA cloning (using plasmids and bacteria) and the amplification often used as a prelude to pyrosequencing. Merely by way of example, DNA related to a portion of the RET gene may be amplified 235. Sequencing 240 results may be clonal results when amplification 238 has been clonal.

As known to persons skilled in the art, DNA sequencing 240 may be performed by Sanger sequencing, pyrosequencing, or using another technique, or a combination of techniques.

In an embodiment of the invention, sequencing 240 is not performed. Merely by way of example, amplification 235 may in some cases provide information for interpretation 150. For example, real-time PCR (e.g. TaqMan) may in some cases provide information for interpretation 150.

In an embodiment of the invention, RNA is extracted, amplified, and analyzed. As known to persons skilled in the art, a variant of PCR is available for RNA.

In cases where the person of interest is a Table 2 person and analysis 140 (or similar step) yields a variant (as compared to wild type) that is as yet unassociated with an MH/LD syndrome, it would not be unreasonable to assume that the variant is associated with an MH/LD syndrome in Table 2 persons, given that (a) MEN2B and other MH/LD syndrome(s) are rare, (b) the non-biochemically-based evidence for MEN2B in Abraham Lincoln as presented in The Physical Lincoln, and (c) a lack of evidence that the variant is a normal variant. Various steps may be taken to acquire evidence that increases the probability that the variant is associated with MEN2B or an MH/LD syndrome. Merely by way of example, such steps include but are not restricted to studies of the activity of a variant RET protein, and studies on persons who have MEN2B. In addition, if the same variant were found in multiple bioartifacts putatively associated with one or more Table 2 people, then that would ordinarily increase the confidence that the variant was associated with MEN2B or another MH/LD syndrome in Table 2 persons. Thus, evidence from such steps may aid in the interpretation 150 and/or revising 180.

As taught above, an association (or non-association) of artifact 100's bio-component 110 with an MH/LD syndrome may be used 180 to revise the probability 170 that a claim 160 about artifact 100 is true. For positive claims (e.g. a claim that artifact 100 is associated with a Table 2 person or candidate Table 2 person), if the association is present, then the probability 170 of the claim's truth is generally revised upwards. For positive claims, if the association is absent, then the probability 170 of the claim's truth is generally revised downwards. For negative claims (e.g. “the artifact is not associated with President Lincoln”), the revisions are generally opposite in direction of those just mentioned if the association is the same.

The magnitude of the adjustment in probability may depend on several factors, including, but not restricted to, technical aspects associated with processing the bio-component, and the certainty of the results of the analyses. In general, the more certain the results of analysis 140, and the more certain the association between the analysis result and an MH/LD syndrome, the greater the magnitude of the probability adjustment, all other factors being equal.

The probability 170 of claim 160 being true can be, for some claims, equated with a claim that the artifact 100 is authentic, i.e artifact 100 is associated with a person of interest according to the claim. Thus, the invention may have application as a means to authenticate artifacts associated with Table 2 persons. The invention may be used to positively authenticate a bioartifact (i.e. associate the artifact more strongly with a person of interest) or negatively authenticate a bioartifact (i.e. associate the artifact less strongly with a person of interest).

In an embodiment of the invention, claim 160 may concern whether a person is related by blood to a Table 2 person. Merely by way of example, if a bioartifact were discovered that was confidently associated with a man hypothesized to be Nancy Hanks Lincoln's father, then finding an MEN2B-associated variant in the bio-component of the bioartifact could be used as evidence to increase the probability that the man was indeed the father of Nancy Hanks Lincoln.

In some cases, it may not be possible to determine, from biochemical analysis alone, which Table 2 person is associated with an artifact. Additional analyses have the potential to help in this situation. For example, as Table 2 currently stands, analyses that identified the biochemical material as originating from a female would increase the probability that Nancy Hanks Lincoln was the source of the biochemical material, and decrease the probability that Abraham Lincoln was the source. As an additional example, Abraham Lincoln's reported blood type (supra.) could be similarly employed.

DNA is not the only biochemical material that may be employed in the invention. Other possible materials include, but are not restricted to RNA and protein. Merely by way of example, a variant in DNA that is associated (or suspected to be associated) with MEN2B may have corresponding variants in RNA (e.g. messenger RNA) or in a protein. For example, a variant DNA nucleotide may lead to a variant messenger RNA nucleotide, and a variant messenger RNA nucleotide may lead to a variant amino acid residue in a protein. Thus, detecting a corresponding variant in RNA or protein would often have the same effect as detecting a variant in DNA.

It is also possible, in principle, that MEN2B in Table 2 persons is associated with a variant in RNA metabolism. For example, it is generally acknowledged that MEN2B is associated with increased activity of the RET protein. One way to get increased overall RET activity is for a plurality of protein molecules to be more active in their function. (This has been reported in association with MEN2B.) Another way is for protein molecules of normal activity to be present in supra-normal quantity. Additional ways may be possible. Thus, a variant in RNA metabolism in which more RET protein were synthesized might lead to the clinical syndrome of MEN2B. (Or it might not, depending on the particulars.) Other variants in RNA metabolism are also possible, e.g. splicing variants.

Similarly, variants in protein metabolism could also, in principle, yield increased overall RET activity.

In addition, a variant in metabolism “downstream” of RET could, in principle, have approximately the same effect as increased RET activity, i.e. a variant could be present in a metabolic pathway that propagates the effect of RET activity.

Detecting a variant in RNA metabolism, RNA structure, protein metabolism, protein structure, or a downstream RET pathway could, therefore, be incorporated into the invention.

Once a single bioartifact has been authenticated in its association with a Table 2 person (by virtue of its positive relation to MEN2B or other MH/LD syndrome) to a high level of confidence, additional analysis of biochemical material in that bioartifact may lead to the discovery of other biochemical features (herein “derived features”) that could be used in authenticating artifacts claimed to be associated with Table 2 persons. Merely by way of example, if an artifact is authenticated as containing biochemical material from President Lincoln using the invention, then further analysis of biochemical material from the same artifact may produce information about other bio-markers that can be confidently associated with Lincoln. Such markers include, but are not restricted to HLA types, or a panel of single nucleotide polymorphisms, or a pattern of tandem repeats.

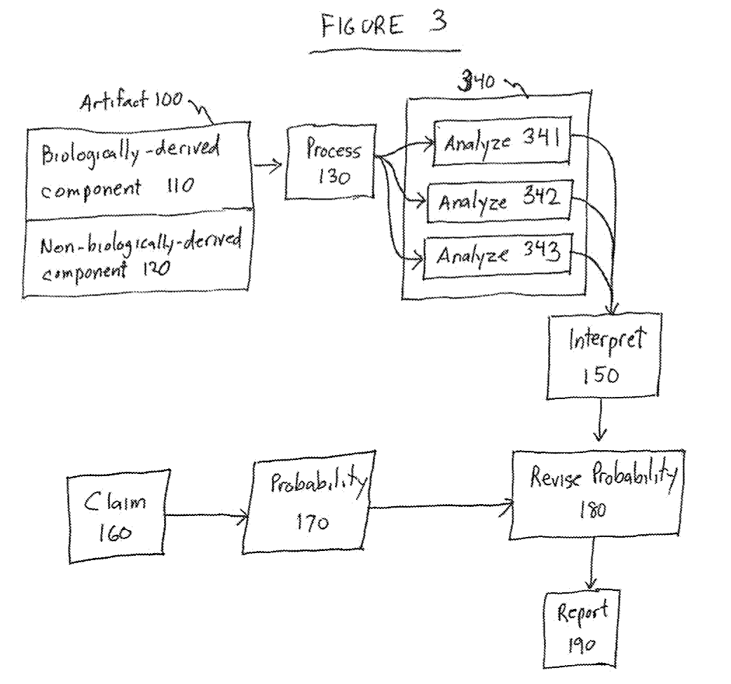

FIG. 3 shows an embodiment of the invention. Items 100, 110, 120, 130, 160, 170, 180, and 190 are as in FIG. 1. Analysis 340 may be based on sub-analysis 341, sub-analysis 342, sub-analysis 343, or a combination thereof. Sub-analysis 341 is based on biochemical information related to an MH/LD syndrome. Sub-analysis 342 is based on biochemical information related to derived features. Sub-analysis 343 is based on other biochemical information (e.g. biochemical correlates of sex, blood type, or other phenotypic features not ascertained as a derived feature or as a feature of an MH/LD syndrome). Sub-analysis 341 and/or sub-analysis 342 must be performed, but sub-analysis 343 need not be. Sub-analyses may be performed in parallel or sequentially. Results of one sub-analysis may provide information for choosing a subsequent sub-analysis. A given sub-analysis may be performed more than once.

Having multiple sub-analyses 341 342 343 may in some cases make it more difficult to fake a result. Merely by way of example, a counterfeiter may obtain blood from a contemporary person who has the same genetic variant related to an MH/LD syndrome as Abraham Lincoln. (This may not be easy, given the rarity of MEN2B.) Adding this blood to an artifact could yield an erroneous conclusion about the authenticity of the artifact if only sub-analysis 341 is performed. If additional sub-analyses 342 343 are performed, then in some cases the counterfeiter may have to find a person who also matches the features of Lincoln in these sub-analyses, which would likely be a more difficult task than finding a match for sub-analysis 341 only.

None of the Table 2 persons are known to have lived beyond 1871. It appears that no other cases of MEN2B predate them, as reported in the medical literature. In an embodiment of the invention, two or more people entity records are stored in a computer-readable memory, the two or more people entity records each comprising identity information and MH/LD-syndrome information from a human-derived artifact dated earlier than 1872. The two or more people entity records are used to revise the probability that the human-derived artifact is associated with a person of interest. Merely by way of example, the person of interest may be a Table 2 person.

The invention has several advantages over the existing art, including, but not restricted to:

(1) An embodiment requires a small length of DNA. The variants listed in Table 3 span only one or a few codons. This is typically an advantage when factors such as DNA degradation, limited supply, or limited access are considered.
(2) An embodiment of the invention is based on DNA variations that are very rare in the general population. This is typically an advantage when contamination is a possible factor. For example, if MEN2B has a prevalence of about one in a million in the general population, then the odds remain remote that any of 10 more-or-less-randomly selected people who hypothetically contaminate a Lincoln bioartifact will have an MEN2B-associated biochemical variant that could masquerade as a positive association with a Table 2 person.
(3) The need for a sample that is unequivocally known to derive from Lincoln, which can serve as a standard for subsequent analyses, is substantially reduced. The Physical Lincoln presents a case for diagnosing an MH/LD syndrome (MEN2B) in Lincoln, based on non-biochemical data. Given the rarity of MEN2B in the general population, finding an MEN2B-related biochemical variant in a bioartifact having only a moderate pre-analysis probability of being derived from Lincoln would be reasonably interpreted as authenticating the bioartifact. In some cases, this artifact could then become a standard. (It is noted that physicians diagnosed MEN2B before the availability DNA-based methods for doing so).

It should be noted that the above sequence of steps is merely illustrative. Any of the above steps can also be separated or combined, depending upon the embodiment. In some cases, the steps can also be changed in order without limiting the scope of the invention claimed herein. One of ordinary skill in the art would recognize many other variations, modifications, and alternatives. It is also understood that the examples and embodiments described herein are for illustrative purposes only and that various modifications or changes in the light thereof will be suggested to persons skilled in the art and are to be included within the spirit and purview of this document.

TABLE 1
Features of MEN2B in President Abraham Lincoln

| | | Page(s) in |
|---|---|---|
| | | The Physical |
| MEN2B Feature | Lincoln Feature | Lincoln |
| Large lips | Large lower lip | 128-135 |
| Bumpy lips | Bumps on lips | 128-135 |
| Marfanoid habitus | Marfanoid habitus | 44-81, 117- |
| | | 121 |
| Gastrointestinal | Constipation | 137-139 |
| symptoms | | |
| Endocrine cancer | Weight loss apparently | 146-163, 220- |
| | beginning circa 1860; | 233 |
| | Declining health in | |
| | later years; Symptoms | |
| | of pheochromocytoma | |
| | in 1865 | |
| Family history of | Suspicion of MEN2B in | 140-145, 106- |
| MEN2B in | Lincoln's mother and | 115 |
| autosomal | three of his sons | |
| dominant pattern | | |
| Asymmetric skull | Asymmetric skull | 194-205 |
| Conjunctival | Conjunctival masses | 174 |
| masses | | |
| Early death | Death from gunshot | |
| | wound at age 56 | |

TABLE 2
Blood Relatives of President Lincoln's Biological Mother
Currently Having a High Probability of Having Had MEN2B

Nancy Hanks Lincoln (the President's biological mother)
President Abraham Lincoln
Edward Lincoln (the President's second son)
William Lincoln (the President's third son)
Thomas “Tad” Lincoln (the President's fourth son)
Thomas(?) Lincoln (the President's younger brother)
The father of Nancy Hanks Lincoln (whoever he may have been)

TABLE 3
Variants of the RET Gene Reported in MEN2B

| Variant | Exon | Reference |
|---|---|---|
| Y791F | 13 | Dvorakova et al. Exp Clin Endocrinol Diabetes. 2006; |
| M918T | 16 | 114: 192-196. |
| V804M | 14 | Cranston et al. Cancer Res. 2006; 66: 10179-10187. |
| E805K | 14 | |
| V804M | 14 | Miyauchi et al. Jpn J Cancer Res. 1999 Jan; 90(1): |
| Y806C | 14 | 1-5. |
| V804M | 14 | Menko et al. J Clinical Endocrinology Metabolism. |
| S904C | 15 | 2002; 87: 393-397. |
| A883F | 15 | Gimm et al. J Clinical Endocrinology Metabolism. |
| | | 1997; 82: 3902-3904. |
| M918T | 16 | Eng et al. JAMA. 1996; 276: 1575-1579. |
| M918T | 16 | Kitamura et al. Hum Mol Genet. 1995; 4: 1987-1988. |
| S922Y | 16 | |
| S922Y | 16 | Hoff et al. Ann Rev Physiol. 2000; 62: 377-411. |

Note 1:
Each row lists a variant in the RET gene that has reportedly been associated with MEN2B in human(s). The “Variant” column lists the amino acid substitution(s) and codon number(s). When two amino acid substitutions are given, they generally occur on the same allele (i.e., cis). The “Exon” column supplies the RET exon number(s) on which the amino acid substitution(s) have been reported.
Note 2:
Hoff & Gagel teach that approximately one new MEN2 DNA variant per year is discovered (A. O. Hoff, R. F. Gagel. “Multiple endocrine neoplasia type 2.” Pages 3533-3550 (Chapter 192) in: L. J. DeGroot, J. L. Jameson (eds). Endocrinology. 5th ed. Philadelphia: Elsevier-Saunders, 2006.).

1. A method for positively or negatively authenticating one or more bioartifacts claimed for a selected human entity, the method comprising:
identifying an artifact having a biologically-derived component, the biologically-derived component having a first probability of being derived from a person of interest, the person of interest being a blood relative of President Abraham Lincoln's biological mother who is free from substantial counterpoints to dx of MEN2B syndrome has a high probability of having an MEN2B syndrome;
processing one or more portions of the artifact to provide an analyzable mass derived from one or more portions of the biological component;
analyzing the analyzable mass derived from the one or more portions of the biological component;
determining whether the analyzable mass derived from the one or more portions of the biological component is associated with one or more target molecule fragments, the one or more target molecule fragments being indicative of at least the MEN2B syndrome in a human; and
using the determining of the analyzable mass to provide a second probability of the biological component of the artifact being derived from the person of interest.
